(19) United States
(12) Patent Application Publication
(10) Pub. No.: US 2002/0182647 A1
(43) Pub. Date: Dec. 5, 2002

(54) Histoscreen method

(76) Inventor: Michael R. Emmert-Buck, Silver Spring, MD (US)

Correspondence Address: Peter A. Borsari, Esq., Borsari & Associates, P.C., Suite 206, 2001 Jefferson Davis Highway, Arlington, VA, 22202, US

(21) Appl. No.: 10/134,392

(22) Filed: Apr. 30, 2002

Related U.S. Application Data
(60) Provisional application No. 60/287,019, filed on Apr. 30, 2001.

(51) Int. Cl. G01N 33/53

(52) U.S. Cl. 435/7.2

(57) ABSTRACT

A method for analyzing biomolecules, such as DNA, mRNA and proteins, in tissue samples, including tissue samples in which these biomolecules are expressed at moderate or low levels of abundance. The method allows multiple tissue samples to be analyzed simultaneously and can be utilized for fresh/frozen tissue samples as well as archival formalin-fixed, paraffin-embedded tissue specimens. The method comprises determining the cellular content of a tissue block, obtaining a plurality of small tissue sections from the tissue block in such a manner that each of said plurality of small tissue section contains at least one cell type, homogenizing each of said plurality of tissue sections in a buffer to create a homogenized specimen, applying said homogenized specimen onto a membrane in order to produce a HistoStamp, applying a labeled probe that is specific for an individual biomolecule corresponding to said at least one cell type to said HistoStamp, and measuring the signal from said labeled probe to determine the abundance level of said biomolecule.

### RELATED APPLICATION

This application claims the benefit of priority of U.S. provisional application Serial No. 60/287,019, filed in the United States Patent & Trademark Office on Apr. 30, 2001 in the name of Michael R. Emmert-Buck.

### FIELD OF INVENTION

The present invention relates to the field of tissue cell analysis. More specifically, the present invention relates to a method for analyzing DNA, mRNA and proteins in tissue samples, including tissue samples wherein these biomolecules are expressed at moderate or low levels of abundance. The method of the present invention allows multiple tissue samples to be analyzed simultaneously and can be utilized for fresh/frozen tissue samples as well as archival formalin-fixed specimens.

### BACKGROUND OF THE INVENTION

With the completion of the Human Genome Project (HGP), a new era of biomedical research is at hand. A primary challenge at this post-sequencing stage is being called “functional genomics”, that is, the effort to determine the physiological role of every human gene. An important first step in the process of discovering the function of each gene is to carefully measure the expression patterns of mRNA transcript and proteins in human tissue specimens. By measuring specific expression patterns, one can determine the particular normal cell types in which each gene is expressed and also can determine the particular abundance level that each gene is expressed.

Equally important in determining the physiological role of every human gene is the measurement of the expression patterns of diseased cells. Determination of the genes and gene products that are dysregulated in pathological conditions will lead to the development of new diagnostic and therapeutic strategies.

Currently, the standard methods for studying mRNA and protein levels in tissue samples are immunohistochemistry (IHC) and in-situ hybridization (ISH). A recently developed, high-throughput variation on these methods is called “tissue arrays”, where multiple, small tissue samples are gridded on a glass slide and studied in parallel. Whether utilizing the single specimen approach or the tissue arrays approach, both the IHC method and the ISH method have two important limitations. First, each of these methods provide only semiquantitative measurement of mRNAs and protein levels. Second, both methods only are capable of measuring mRNAs and proteins that are expressed at high abundance levels. However, they are severely limited in their ability to detect biomolecules that are expressed at lower levels of abundance. An analysis of human mRNA and proteomic expression databases by the present inventor indicates that the majority of the new genes identified by the Human Genome Project are expressed at moderate or low abundance levels. Thus, it is difficult, if not impossible, to measure mRNAs and proteins using either the IHC method or the ISH method.

In addition, the vast majority of human tissue samples that have been collected in hospitals throughout the world for the past forty to fifty years, commonly referred to as archival samples, have been processed through a formalin-fixation application. That is, these tissue samples have been fixed using cross-linking fixatives, such as formalin. This fixation method has found wide success for analyzing tissue samples in the traditional microscopic analysis of histologic sections for patient diagnosis. However, the formalin-fixation application induces extensive cross-linking of the biomolecules contained in the tissue samples, thereby rendering the tissue samples of little value for many molecular analysis methods that have been developed in recent years.

Consequently, a need exists for a new method for detecting and measuring biomolecules in various tissue samples. Such a method should enable the detection of biomolecules which are expressed at moderate to low levels of abundance. In addition, such a method should be applicable to both fresh/frozen tissue samples and to formalin-fixed tissue samples. Moreover, such a method for analyzing tissue samples should provide increased flexibility of specimen selection and should allow multiple samples to be analyzed simultaneously.

### SUMMARY OF THE INVENTION

Accordingly, it is an object of the present invention to provide a method of analyzing tissue samples for detecting biomolecules, including DNA, mRNA and proteins.

It also is an object of the present invention to provide a method of analyzing tissue samples to detect biomolecules that are expressed at moderate or low levels of abundance.

It is an additional object of the present invention to provide a method of analyzing tissue samples to detect biomolecules in tissue samples that are fresh/frozen tissue samples.

It is another object of the present invention to provide a method of analyzing tissue samples to detect biomolecules in tissue samples that are archival formalin-fixed specimens.

It is a further object of the present invention to provide a method of analyzing tissue samples that allows multiple tissue samples to be analyzed simultaneously.

It is yet another object of the present invention to provide a method of analyzing tissue samples that provides increased flexibility of specimen selection over previously used tissue array formats.

Additional objects, advantages and novel features of the invention will be set forth in part of the description and claims which follow, and in part will become apparent to those skilled in the art upon examination of the following specification or may be learned by practice of the invention.

### BRIEF DESCRIPTION OF THE DRAWINGS

The present invention will be better understood with reference to the appended drawing sheets, wherein:

FIG. 1 is a flow chart illustrating the method of the present invention using a frozen tissue block.

FIG. 2 is a flow chart illustrating the method of the present invention using a formalin-fixed tissue block.

FIG. 3 shows a histology slide prepared from sectioning a formalin-fixed, paraffin-embedded tissue block.

FIG. 4 shows a paraffin tissue block corresponding to FIG. 3, after removal of tissue punches.

FIG. 5 shows a paraffin HistoStamp tissue block with the placement of two different sized tissue punches into the block.

FIG. 6 is a microscopic view of normal colon epithelium containing cells that stain positive (darkly colored) for cytokeratin.

FIG. 7 is a microscopic view of colon cancer containing cells that stain positive (darkly colored) for cytokeratin.

FIG. 8 is a microscopic view of normal pancreatic stroma that does not contain cells that are positive for cytokeratin.

FIG. 9 shows a plurality of tissue punches of normal colon epithelium prepared from the HistoStamp block that are cut at various thicknesses and placed onto glass slides.

FIG. 10 shows a plurality of tissue punches of colon cancer from the HistoScreen block that are cut at various thicknesses and placed onto glass slides.

FIG. 11 is a measurement of cytokeratin protein levels in normal colon epithelium, colon cancer, and normal pancreatic stroma.

FIG. 12 is a graphical representation of cytokeratin protein levels in normal colon epithelium.

FIG. 13 is a graphical representation of cytokeratin protein levels in colon cancer.

FIG. 14 is a graphical representation of cytokeratin protein levels in normal pancreatic stroma.

### DETAILED DESCRIPTION

The present invention relates to a method of analyzing tissue samples for detecting biomolecules, particularly DNA, mRNA and proteins. More particularly, the present invention provides a method of analyzing tissue samples for detecting biomolecules that are expressed at moderate or low abundance levels. The inventive method, hereinafter sometimes referred to as the “HistoScreen method” can be utilized for tissue samples that are fresh, frozen, or processed tissue that has not been extensively cross-linked. In addition, a modification of this method allows for the analysis of tissue samples that have been fixed in cross-linking fixatives, such as archival formalin-fixed, paraffin-embedded (FFPE) tissue samples.

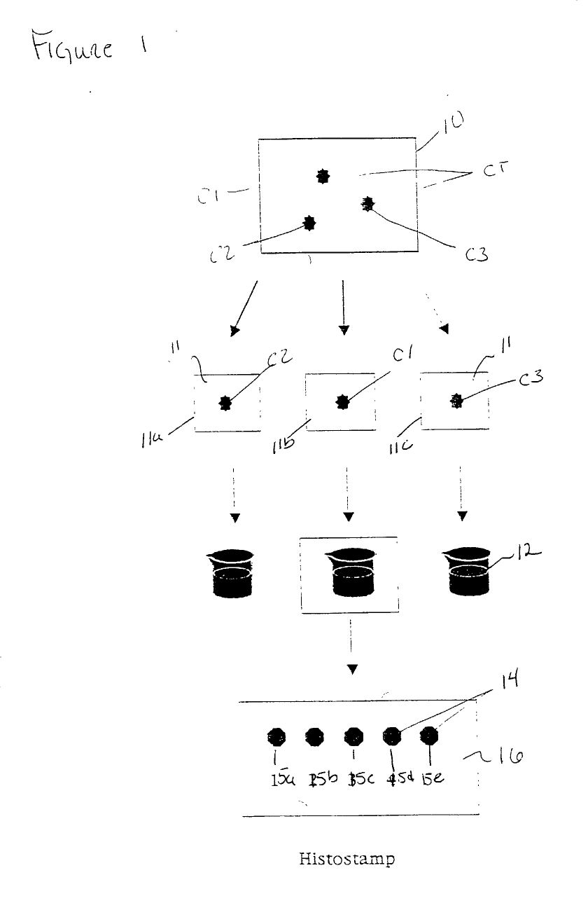

Referring to FIG. 1, in the method of the present invention, the cellular contents of a tissue specimen initially are determined by sectioning a tissue block 10 with a microtome and preparing a standard histology slide for microscopic visualization. Microscopic analysis of the histology slide indicates the tissue block locations of normal epithelium (N), stroma (S), and tumor (T). Based on the microscopic evaluation, the tissue block 10 is then physically cut or punched into a plurality of small pieces 11 such that each individual piece contains one or more specific cell types CT of interest, for example normal (N), stroma (S) and tumor (T). Although only three tissue pieces and three cell types are shown, it is to be understood that the tissue block can be cut into any suitable number of small pieces and that the three pieces 11a, 11b, 11c shown in FIG. 1 are merely for the purpose of illustrating the present invention. In addition, it is to be understood that the tissue sample may contain only one cell type or multiple cell types and that the three cell types C1, C2, C3 shown are merely illustrative. Thus, any suitable number of small pieces 11 and cell types CT are contemplated to be within the scope of the present invention. For example, a tissue block from a patient with cancer may be subdivided into a plurality small pieces that contain different cell types of interest such as low-grade tumor, high-grade tumor, normal glands, and the like. Once the plurality of tissue pieces have been selected, each tissue piece 11a, 11b, 11c is homogenized in a buffer solution 12 in order to produce a cellular lysate 14 containing the particular cell type(s) CT. In this manner, a lysate containing the cellular biomolecule(s) of interest is obtained.

Once the cellular lysate 14 has been obtained, the lysate is applied onto a surface 16, an example of which is a nitrocellulose membrane. The surface (e.g. nitrocellulose membrane) typically is a square measuring approximately 0.5 cm on each side and hereinafter may sometimes be referred to hereinafter as a “HistoStamp”. However, it is to be understood that the HistoStamp can be of any size and shape and does not have to be limited to a 0.5 cm square. The cellular lysate 14 is applied onto the surface as multiple small “spots” each having a different dilution strength. The number of multiple spots may be any suitable number that allows a dilution curve to be obtained. Five spots 15a, 15b, 15c, 15d, 15e are shown in FIG. 1 and are intended to be illustrative of the present invention. It is important that the cellular lysate 14 is applied to the HistoStamp starting with a highly concentrated solution, spot 15a, followed by serial dilutions of decreasing lysate concentration, that is, spots 15b, 15c, 15d, 15e. Each small tissue piece 11 is capable of creating a sufficient number of cellular lysates 14 to prepare multiple HistoStamps, in the range of about one hundred to about two hundred (100-200) HistoStamps per tissue piece.

The abundance level of a biomolecule of interest, for example a protein, DNA, or mRNA species, is determined by applying a labeled probe that is specific for the particular individual biomolecule of interest. The signal from the probe is measured, thus providing a determination of the abundance level of the biomolecule in the tissue sample. The highly concentrated lysate, spot 15a will permit low abundance proteins and mRNA transcripts to be measured. These proteins and mRNAs cannot be analyzed using standard IHC or ISH hybridization (or the tissue array version of these techniques) since they are too low in abundance. The spots of decreasing lysate concentration, spots 15b, 15c, 15d, 15e, represent a dilution curve that permits more accurate and quantitative calculation of DNA/mRNA/protein abundance levels than analysis of a single concentration of the tissue lyrate.

A modification of the HistoScreen method is provided to analyze archival tissue samples. Because of the severe biomolecule crosslinking found in tissue samples which have been archived using a crosslinking fixative application, such as the formalin-fixative application, it is not possible to purify and recover the biomolecules, for example, proteins or mRNA. Rather, the proteins, mRNAs and DNA in the specimens will remain clumped together. Thus, a cellular lysate containing one or more purified biomolecules cannot be produced. As a result, the cellular lysate, or lysate dilutions, can not be applied to a series of HistoStamps.

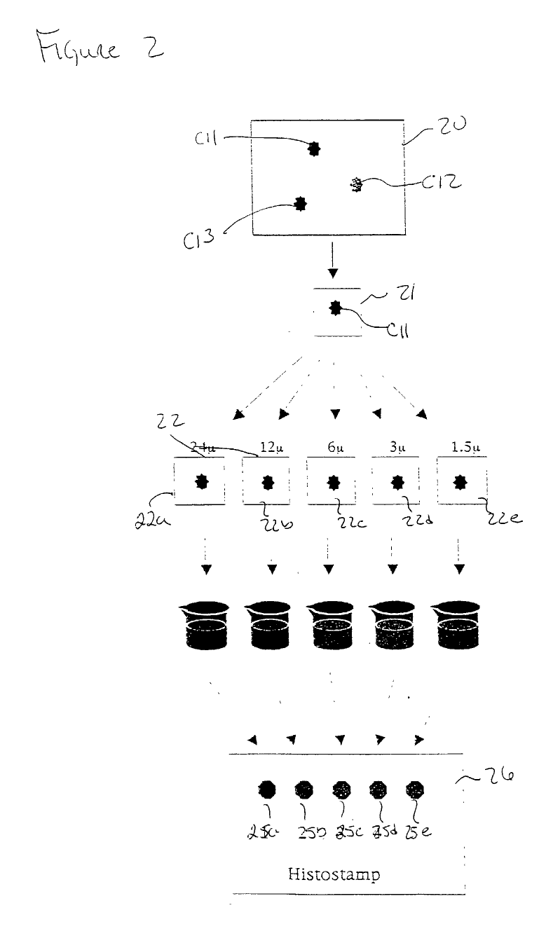

To overcome the problems encountered with tissue samples prepared by the formalin-fixation application, the HistoStamp is created from the actual archived tissue sample. Referring to FIG. 2, the cellular contents of a tissue specimen initially are determined using standard histological slide preparation techniques and microscopic visualization. Based on the microscopic evaluation, the tissue block 20 is grossly cut into a plurality of small pieces 21 such that each individual piece is enriched in one or more specific cell types CT of interest. As illustrated in FIG. 2, the tissue block 20 has three cell types C11, C12, C13 and one small piece 21 is shown having cell type C11. However, it is to be understood that the process depicted in FIG. 2 is merely illustrative and any suitable number of pieces 21 and cell types are contemplated to be within the scope of the present invention.

Each individual small tissue piece 21 is mounted on a microtome and a multiple of serial recut sections 22 are produced. As shown in FIG. 2, five recut sections 22a, 22b, 22c, 22d, 22e are shown, each having a different thicknesses (for example; 24, 12, 6, 3, and 1.5 microns). However, it is to be understood that the tissue piece can be recut into any suitable number of recut sections with varying thickness and that the five recut sections shown in FIG. 2 are merely for the purpose of illustration. Each recut section thereafter is homogenized in a buffer and each recut section is applied as a single spot 25 on a HistoStamp 26. Thus, rather than creating a cellular lysate and applying lysates of serial dilution to the HistoStamp, the several recut sections 22a, 22b, 22c, 22d, 22e, each having a different thicknesses, are each applied as a spot 25a, 25b, 25c, 25d, 25e as shown in FIG. 2. In this manner, the “serial dilution” of spot concentrations on the HistoStamp is created by making recut sections of different thicknesses on the microtome, each of which is wholly applied as a single spot. In addition, a recut section is used periodically to prepare a standard histology slide that can be viewed under the microscope. This allows verification that the cellular contents, that is the cell types of interest, of the tissue do not change as the tissue specimen is sectioned deeper into the block.

As in the previously-described method, a labeled probe is applied the HistoStamp 26 having the multiple spots 25a, 25b, 25c, 25d, 25e and the signal from the probe is measured to determine the abundance level of the biomolecule in the tissue sample. By using this method, a series of HistoStamps can be prepared from formalin-fixed tissue samples, representing a powerful new approach to analyzing biomolecular content from the world's archive of tissue specimens.

A typical HistoScreen protocol of the present invention for the formalin-fixed, paraffin-embedded tissue now will be described. Eight (8) to ten (10) sections from a tissue block are cut and placed on a microscope slide. The sections are treated with histological stain in order to observe and determine the morphological make up of the tissue. FIG. 3 shows such a histology slide prepared from a section of human tissue block. Microscopic analysis of the histology slide indicates the tissue block locations of normal epithelium (N), stroma (S), and tumor (T). Defined cell types from which tissue punches can be taken are marked as shown in FIG. 3.

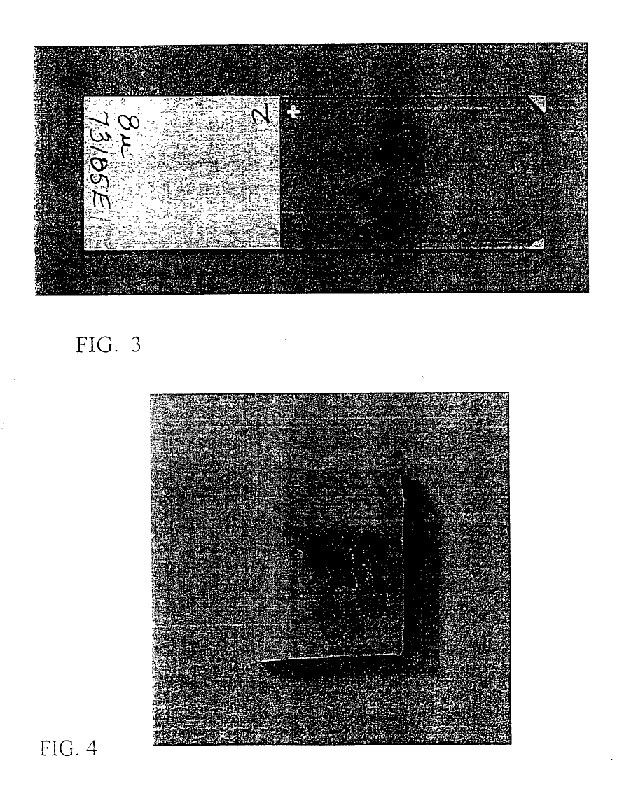

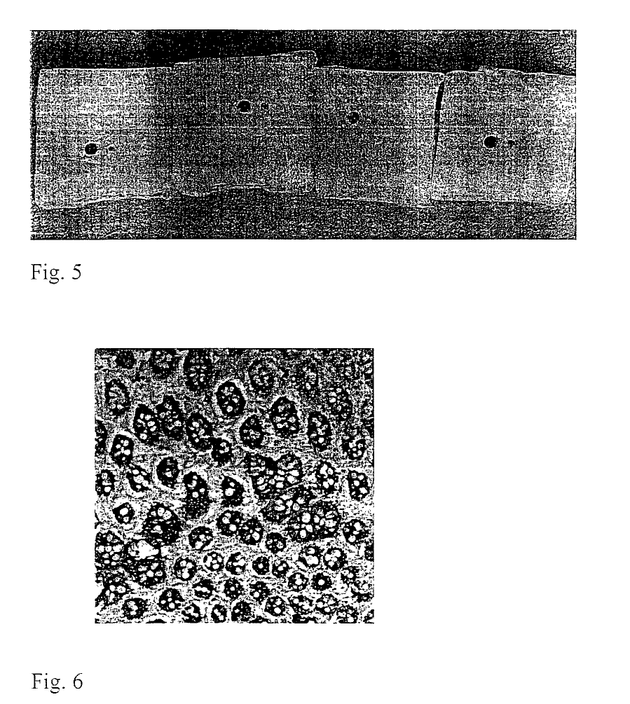

HistoStamp blocks containing the morphologically defined tissue punches from the designated tissue are prepared by removing a tissue punch from the morphologically defined area of the tissue block as shown in FIG. 4. Each tissue punch is placed in a recipient paraffin block as shown in FIG. 5. More particularly, referring to FIG. 5, a paraffin HistoStamp tissue block shows the placement of two different sized tissue punches. Each tissue punch within a block represents a different volume of the same tissue type (e.g., normal epithelium, tumor, or stroma). The tissue punches are selected to be of various sizes in order to vary the amount of tissue that is subsequently processed into a HistoStamp. The paraffin recipient block containing the tissue punch(es) is warmed to soften the paraffin and set the tissue punch(es) into the paraffin of the recipient block.

Sections of varying thicknesses are cut from the paraffin HistoStamp block and homogenized in a buffer solution to prepare the cellular lysate. The resulting lysate is placed on a microscope slide for the HistoScreen assay. The following Table illustrates the preparation of three samples from HistoStamp blocks, each of which has been cut into three different thicknesses in order to provide three dilution strengths.

| Sample | Organ | Morphological Area | Thickness | | Volume mm³ |
|---|---|---|---|---|---|
| 73185-D | colon | normal | 5 | μ | 0.0157 |
| | | | 19 | μ | 0.0471 |
| | | | 30 | μ | 0.0942 |
| 73185-D | colon | tumor | 5 | μ | 0.0157 |
| | | | 15 | μ | 0.0471 |
| | | | 30 | μ | 0.0942 |
| 73185-E | pancreas | stroma | 5 | μ | 0.0157 |
| | | | 15 | μ | 0.0471 |
| | | | 30 | μ | 0.0942 |

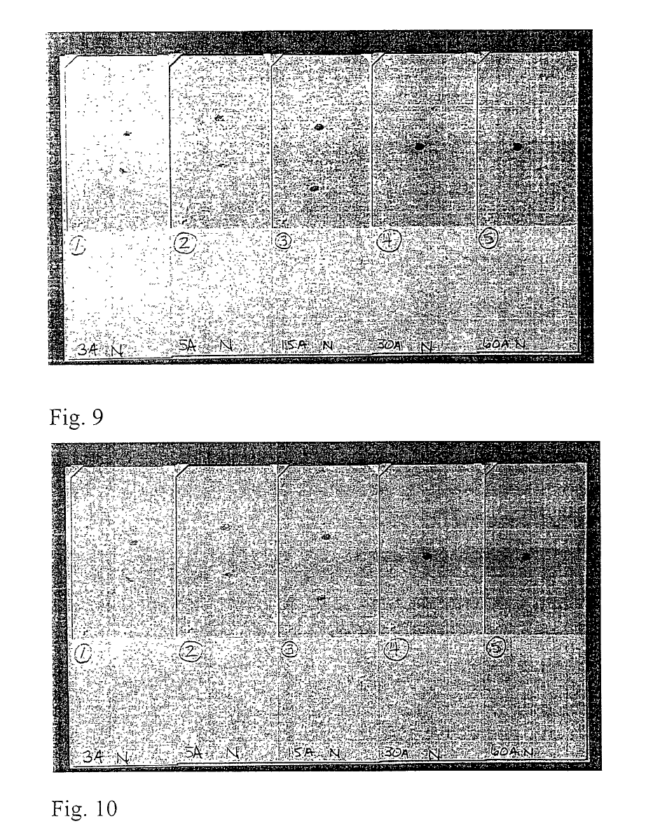

Referring to FIGS. 9 and 10, FIG. 9 shows sections of tissue punches of normal colon epithelium from the HistoScreen block that are cut at various thicknesses and placed onto glass slides. FIG. 10 shows sections of tissue punches of colon cancer from the HistoScreen block that are cut at various thicknesses and placed onto glass slides.

Each of the prepared slides is heated to a temperature of about 58° C. for about thirty (30) minutes to melt the paraffin and adhere the section to the slide. The slides then are incubated in two changes of xylenes for about five (5) minutes per each change, to remove the paraffin. The resulting slides then are incubated in two changes of 100% ethanol for about one (1) minute per each change and air dried.

The air-dried slides are blocked with a 3% buffer solution of bovine serum albumin-phosphate buffered saline (BSA-PBS) for about thirty (30) minutes at room temperature. Excess blocking buffer is removed and a primary antibody probe diluted in 1% BSA is applied to each slide. The slides with the primary antibody are incubated overnight at room temperature, followed by a wash in PBS for about three (3) to five (5) minutes.

A biotinylated secondary antibody probe diluted in 1% BSA then is applied to each slide and incubated for about thirty (30) minutes at room temperature, followed by a wash in PBS for about three (3) to five (5) minutes. A streptavidin-HRP conjugate probe diluted in 1% BSA then is applied to each slide and incubated for about thirty (30) minutes at room temperature, followed by a wash in PBS for about three (3) to five (5) minutes. Each slide then is incubated in a buffer containing appropriate substrate for about ten minutes, washed in distilled water and air dried.

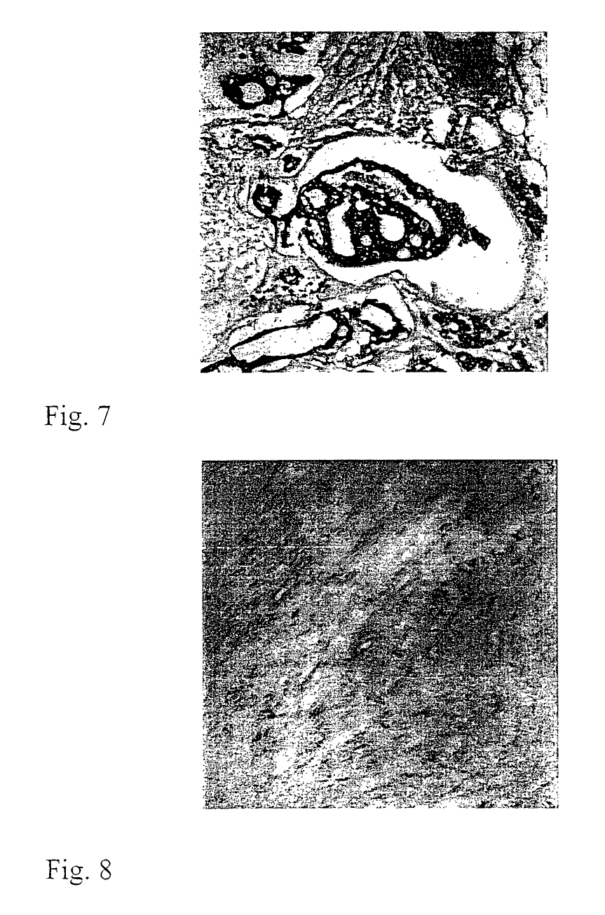

Once the HistoScreen protocol is completed, the probe-treated tissue punches are evaluated. FIGS. 6 and 7 shows a microscopic view of normal colon epithelium containing cells and colon cancer containing cells respectively that stained positive (darkly colored) for cytokeratin, while FIG. 8 shows a microscopic view of normal pancreatic stroma that does not contain cells that are positive for cytokeratin.

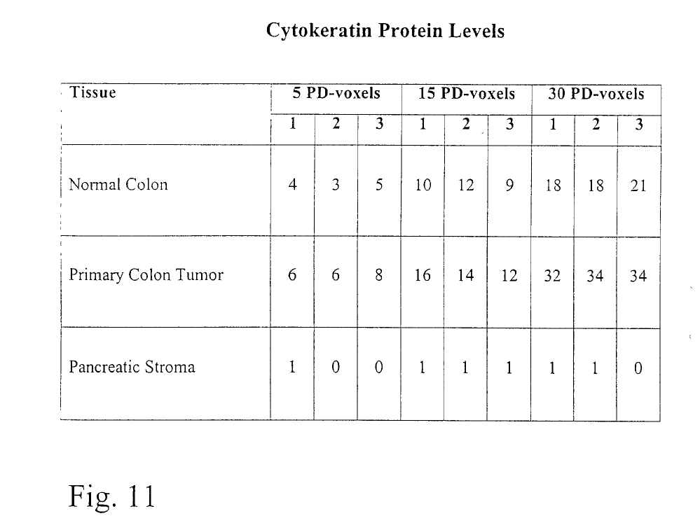

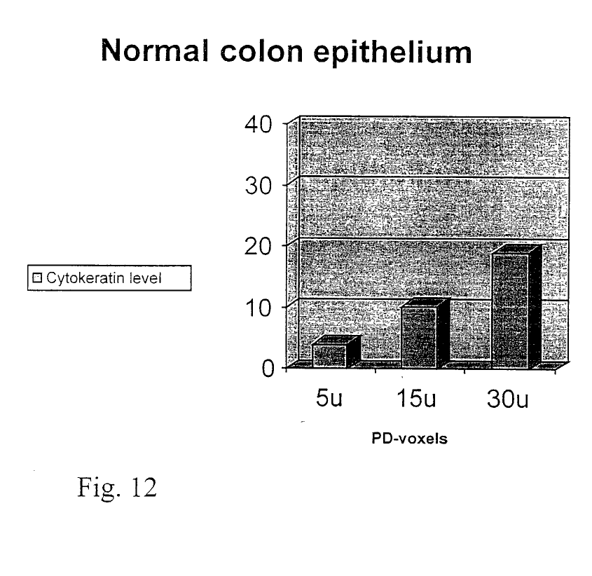

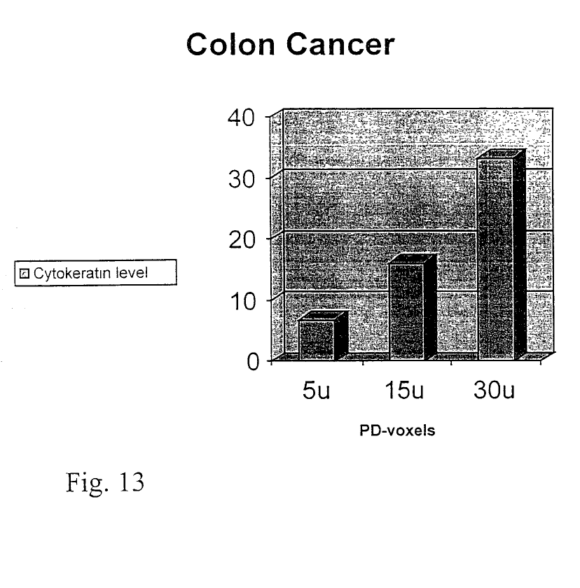

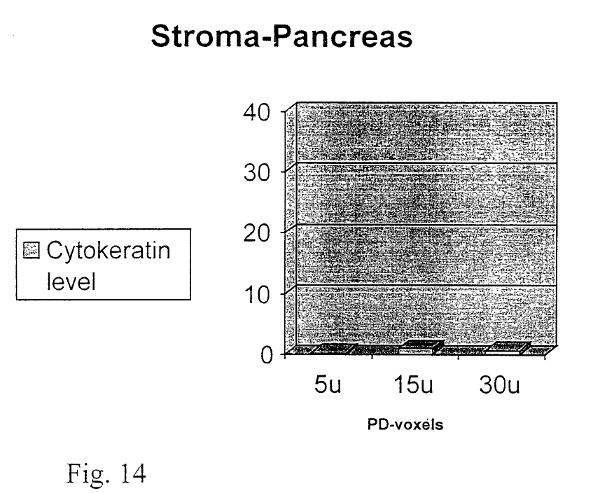

In FIG. 11, a measurement of cytokeratin protein levels in normal colon epithelium, colon cancer and normal pancreatic stroma. Three independent measurements are made for each spot on the HistoStamp, wherein PD-voxels refers to the volume content of each tissue section process onto the HistoStamp. FIGS. 12, 13 and 14 provide graphical representations of the cytokeratin protein levels in normal colon epithelium, colon cancer and normal pancreatic stroma respectively which were prepared from the samples.

The method of the present invention can be used to probe simultaneously multiple tissue specimens. For example, any number or combination of HistoStamps can be placed on a grid and probed, or alternatively, they can be stacked on top of each other and soaked in a buffer containing the labeled probe. The net effect of this method is to permit high-throughput analysis of specific portions of tissue samples, in a highly sensitive and quantitative manner.

The HistoStamp of the present invention also can be utilized in conventional biodetection processes based on the co-production of histology and molecular reagents that are created from the same tissue samples that are utilized for HistoStamps. For example, the HistoStamp can be used to produce a traditional microstamp slide, thereby enabling an investigator to stain or otherwise analyze the tissue using standard methods (e.g. IHC or ISH etc.). In addition, the tissue that is utilized for the preparation of a HistoStamp can be probed for a gene of interest, for example by a Western blot technique. Additional related reagents also can be derived from the tissue utilized for the HistoStamps, including for example, genomic DNA libraries or amplified mRNA populations.

The present invention also contemplates the use of the HistoScreen method with a software program termed “HistoView” that will capture and visualize images, and store and analyze the data generated by the HistoStamps in a particular database. Such software will enable users examining particular biomolecules of interest to review previous studies and append the results of their studies, thereby developing a comprehensive database related to the particular biomolecule.

While particular embodiments of the invention have been described, it will be understood, of course, that the invention is not limited thereto, and that many obvious modifications and variations can be made, and that such modifications and variations are intended to fall within the scope of the appended claims.

What is claimed is:

1. A method for analyzing a tissue sample comprising:
(a) determining the cellular content of a tissue block;
(b) obtaining a plurality of small tissue sections from said tissue block in such a manner that each of said plurality of small tissue section contains at least one cell type;
(c) homogenizing each of said plurality of tissue sections in a buffer to create a homogenized specimen;
(d) applying said homogenized specimen onto a membrane in order to produce a HistoStamp;
(e) applying a labeled probe that is specific for an individual biomolecule corresponding to said at least one cell type to said HistoStamp, and
(f) measuring the signal from said labeled probe to determine the abundance level of said biomolecule.

2. The method in accordance with claim 1, wherein said homogenized specimen is a cellular lysate.

3. The method in accordance with claim 2, wherein said cellular lysate is applied onto said membrane in the form of a spot having a concentration of said cellular lysate.

4. The method in accordance with claim 3, wherein a series of spots of cellular lysate are serially applied onto said membrane in decreasing lysate concentration.

5. The method in accordance with claim 4, wherein an accurate and quantitative calculation of the abundance level of said biomolecule can be obtained by comparing said series of spots of decreasing lysate concentration.

6. The method in accordance with claim 1, wherein a plurality of HistoStamps are disposed on a grid probed simultaneously.

7. The method in accordance with claim 1, wherein a plurality of HistoStamps are stacked and soaked in a buffer containing said labeled probe.

8. The method in accordance with claim 1, wherein said plurality of small tissue sections are a plurality of serial recut sections, each of said recut sections having a different thickness.

9. The method in accordance with claim 8, wherein each of said plurality of serial recut sections are serially applied onto said membrane.

10. A method for analyzing a tissue sample comprising:
(a) determining the cellular content of a tissue block;
(b) obtaining a plurality of small tissue pieces from said tissue block in such a manner that each of said plurality of small tissue pieces contains at least one cell type;
(c) homogenizing each of said plurality of tissue pieces in a buffer to create a cellular lysate;
(d) applying said cellular lysate onto a membrane in the form of a spot having a concentration of said cellular lysate in order to produce a HistoStamp;
(e) applying a labeled probe that is specific for an individual biomolecule corresponding to said at least one cell type to said HistoStamp, and
(f) measuring the signal from said labeled probe to determine the abundance level of said biomolecule.

11. The method in accordance with claim 10, wherein a series of spots of cellular lysate are serially applied onto said membrane in decreasing lysate concentration.

12. The method in accordance with claim 11, wherein an accurate and quantitative calculation of the abundance level of said biomolecule can be obtained by comparing said series of spots of decreasing lysate concentration.

13. The method in accordance with claim 10, wherein a plurality of HistoStamps are disposed on a grid probed simultaneously.

14. The method in accordance with claim 10, wherein a plurality of HistoStamps are stacked and soaked in a buffer containing said labeled probe.

15. A method for analyzing a tissue sample comprising:
(a) determining the cellular content of a tissue block;
(b) obtaining a plurality of serial recut sections from said tissue block in such a manner that each of said plurality of serial recut sections contains at least one cell type and each of said plurality of serial recut sections has a different thickness;
(c) homogenizing each of said plurality of serial recut sections in a buffer;
(d) applying each of said plurality of serial recut sections onto a membrane, each recut section being applied in the form of a spot to produce a HistoStamp;
(e) applying a labeled probe that is specific for an individual biomolecule corresponding to said at least one cell type to said HistoStamp, and
(f) measuring the signal from said labeled probe to determine the abundance level of said biomolecule.

16. The method in accordance with claim 15, wherein each of said plurality of serial recut sections are applied serially to said membrane in the form of a series of spots.

17. The method in accordance with claim 16, wherein an accurate and quantitative calculation of the abundance level of said biomolecule can be obtained by comparing said series of spots of different thicknesses.

18. The method in accordance with claim 15, wherein a plurality of HistoStamps are disposed on a grid probed simultaneously.

19. The method in accordance with claim 15, wherein a plurality of HistoStamps are stacked and soaked in a buffer containing said labeled probe.
